(19) United States
(12) Patent Application Publication
(10) Pub. No.: US 2026/0347767 A1
(43) Pub. Date: Nov. 5, 2026

(54) METHOD FOR SELECTIVE DIFFERENTIATION INTO DORSAL METENCEPHALIC TISSUE FROM EMBRYOID DERIVED FROM PLURIPOTENT STEM CELLS

(71) Applicant: NEXT&BIO INC., Seoul (KR)

(72) Inventors: Tae Hwan Kwak, Gyeonggi-Do (KR); Ji Hun Yang, Seoul (KR)

(73) Assignee: NEXT&BIO INC., Seoul (KR)

(21) Appl. No.: 19/102,724

(22) PCT Filed: Aug. 9, 2023
(86) PCT No.: PCT/KR2023/011789

§ 371 Date: Feb. 10, 2025

(30) Foreign Application Priority Data
Aug. 10, 2022 (KR) 10-2022-0099740

(51) Int. Cl. C12N 5/0793

(52) U.S. Cl. CPC C12N 5/0619; C12N 2501/41; C12N 2501/415; C12N 2506/02

(57) ABSTRACT

The present disclosure relates to a method for selective differentiation into dorsal metencephalic tissue from embryoid bodies derived from pluripotent stem cells, the method comprising the step of selective differentiation into dorsal metencephalic tissue, in which embryoid bodies derived from pluripotent stem cells are cultured in the presence of a dorsal metencephalic tissue induction medium containing a sonic hedgehog (SHh) signaling pathway inhibitor.

### TECHNICAL FIELD

The present disclosure relates to a method for selective differentiation into dorsal metencephalic tissue from embryoid bodies derived from pluripotent stem cells.

### BACKGROUND ART

An organoid, also called ‘organ analogue’ or ‘organ-like body’, is an organ-specific cell aggregate prepared by reaggregating and recombining cells isolated from stem cells or organ-origin cells using a 3D culture method. The organoid contains specific cells of the organ for modeling, reproduces specific functions of the organ, and may be spatially organized in a form similar to an actual organ.

In particular, organoids may be prepared to mimic most human organs using pluripotent stem cells that may differentiate into all cells of the human body, and among these, a brain organoid has received a lot of attention as an in vitro culture model for mimicking various intractable central nervous system diseases to elucidate the pathogenesis and develop treatments.

For example, Lancaster et al. prepared brain organoids using human induced pluripotent stem cells (Non-Patent Document 1). However, the brain organoids prepared in this way have a problem that various organs, such as the cerebrum, midbrain, and retina, are mixed in a single organoid, and their sizes are also different.

Most intractable brain diseases are diseases that occur specifically in a specific brain region, and organoids that have the characteristics of a brain region other than a target disease site or have mixed characteristics of various regions may not accurately mimic brain diseases. Therefore, in order to utilize brain organoids for research on the pathogenesis of intractable brain diseases through modeling, research on developing treatments, etc., it is required to develop a region-specific brain organoid fabrication technology capable of mimicking a specific brain region with high purity.

### PRIOR ARTS

### Non-Patent Document

(Non-Patent Document 1) Madeline A. Lancaster, Juergen A. Knoblich, Generation of Cerebral Organoids from Human Plurpotent Stem Cells, Nat Protoc. 2014 October; 9 (10): 2329-2340.

### DISCLOSURE

### Technical Problem

An object of the present disclosure is to provide a method for selective differentiation into dorsal metencephalic tissue from embryoid bodies derived from pluripotent stem cells, thereby providing a basis for preparing region-specific brain organoids.

However, technical objects of the present disclosure are not limited to the aforementioned purpose and other objects which are not mentioned may be clearly understood to those skilled in the art from the following description.

### Technical Solution

An aspect of the present disclosure provides a method for selective differentiation into dorsal metencephalic tissue from embryoid bodies derived from pluripotent stem cells, the method including a step of selective differentiation into dorsal metencephalic tissue in which embryoid bodies derived from pluripotent stem cells are cultured in the presence of a dorsal metencephalic tissue induction medium containing a sonic hedgehog (SHh) signaling pathway inhibitor.

### Advantageous Effects

According to the present disclosure, it is possible to induce differentiation into dorsal metencephalic tissue from embryoid bodies by the method for selective differentiation into dorsal metencephalic tissue from embryoid bodies derived from pluripotent stem cells. Accordingly, it is possible to provide a basis for preparing a specific brain organoid by inducing selective differentiation into the pons and cerebellum.

### DESCRIPTION OF DRAWINGS

FIG. 1 is a schematic diagram showing a differentiation mechanism of a method for selective differentiation into dorsal metencephalic tissue from embryoid bodies derived from pluripotent stem cells according to one embodiment of the present disclosure.

FIG. 2 shows results of expression of dorsal-metencephalic region-specific genes according to a concentration of an SHh signaling pathway inhibitor in a dorsal metencephalic tissue induction medium of Example.

FIG. 3 shows a developmental state of an embryoid on day 7 after selective differentiation into dorsal metencephalic tissue according to a concentration of the SHh signaling pathway inhibitor in the dorsal metencephalic tissue induction medium of Example.

### BEST MODE

Hereinafter, the present disclosure will be described in detail.

The present disclosure may have various modifications and various embodiments and specific embodiments will be illustrated in the drawings and described in detail in the detailed description.

However, the present disclosure is not limited to specific embodiments, and it should be understood that the present disclosure covers all the modifications, equivalents and replacements included within the idea and technical scope of the present disclosure. In describing the present disclosure, when it is determined that a detailed description of related known arts may obscure the gist of the present disclosure, the detailed description will be omitted.

Terms used herein are used only to describe specific embodiments, and are not intended to limit the present disclosure. A singular expression includes a plural expression unless otherwise defined differently in a context.

In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations of these, in advance.

An embodiment of the present disclosure provides a method for selective differentiation into dorsal metencephalic tissue from embryoid bodies derived from pluripotent stem cells, the method including a step of selective differentiation into dorsal metencephalic tissue, in which embryoid bodies derived from pluripotent stem cells are cultured in the presence of a dorsal metencephalic tissue induction medium containing a sonic hedgehog (SHh) signaling pathway inhibitor.

According to one embodiment of the present disclosure, through the step of selective differentiation into the dorsal metencephalic tissue, the embryoid bodies formed from the pluripotent stem cells may differentiate into a cell aggregate that mimics dorsal-metencephalic characteristics with high purity. Specifically, the cell aggregate mimicking the dorsal-metencephalic characteristics may be an organoid mimicking the dorsal-metencephalic characteristics with high purity. The cell aggregate or organoid mimicking the dorsal-metencephalic characteristics with high purity may mean a state in which the mixing of tissues exhibiting characteristics of other brain regions such as the cerebrum, striatum, and midbrain is minimized.

According to one embodiment of the present disclosure, the step of selective differentiation into the dorsal metencephalic tissue may uniformly induce patterning into dorsal metencephalic tissue throughout the embryoid bodies, while inducing differentiation of the embryoid bodies into the neuroectoderm.

According to one embodiment of the present disclosure, the embryoid bodies derived from the pluripotent stem cells may be embryoid bodies differentiating into the metencephalic tissue. Specifically, the step of selective differentiation into the dorsal metencephalic tissue may induce the embryoid bodies differentiating into the metencephalic tissue such as the pons and cerebellum to be specified as dorsal metencephalic tissue during the differentiation process to have higher cerebellar specificity.

According to one embodiment of the present disclosure, the step of selective differentiation into the dorsal metencephalic tissue may include a step of adding the dorsal metencephalic tissue induction medium to a medium in which the embryoid bodies derived from the pluripotent stem cells are being cultured. Specifically, the medium in which the embryoid bodies derived from the pluripotent stem cells are being cultured may include a metencephalic tissue-inducing medium. Specifically, the step of selective differentiation into the dorsal metencephalic tissue may be performed in an overlapping manner with the treatment of the embryoid bodies during differentiating into specific tissues. More specifically, the step of selective differentiation into the dorsal metencephalic tissue may be performed in an overlapping manner with a step of selective differentiation into metencephalic tissue using the metencephalic tissue-inducing medium. At this time, the metencephalic tissue-inducing medium may include an endogenous WNT secretion inhibitor and a WNT signaling agonist.

According to one embodiment of the present disclosure, the SHh signaling pathway inhibitor may be a material that serves to inhibit the SHh signaling pathway that is naturally activated in some cells within the embryoid bodies. The SHh signaling pathway inhibitor may inhibit the activation of the heterogeneous and uncontrollable SHh signaling pathway by inhibiting the activation of the SHh signaling pathway by means of a protein that generates an SHh signal spontaneously expressed within the embryoid bodies. That is, the embryoid bodies may be selectively and homogeneously differentiated into dorsal metencephalic tissue by inhibiting the endogenous SHh signal by the SHh signaling pathway inhibitor.

According to one embodiment of the present disclosure, the SHh signaling pathway inhibitor may include at least one selected from the group consisting of cyclopamine, itraconazole, robotnikinin, cerulenin, and GANT61. Specifically, the SHh signaling pathway inhibitor may be cyclopamine.

According to one embodiment of the present disclosure, the dorsal metencephalic tissue induction medium may include an SHh signaling pathway inhibitor in a concentration range that exhibits an effect equivalent to the effect exhibited by cyclopamine in a concentration range of more than 10 μM and 30 μM or less. Specifically, the dorsal metencephalic tissue induction medium may include a SHh signaling pathway inhibitor in a concentration range that exhibits an effect equivalent to the effect exhibited by cyclopamine in a concentration range of more than 10 μM and less than 30 μM, 12 μM or more and less than 30 μM, 12 μM or more and 30 μM or less, 15 μM or more and 25 μM or less, or 20 μM or more and 25 μM or less. More specifically, the dorsal metencephalic tissue induction medium may include the SHh signaling pathway inhibitor at a concentration that specifically expresses at least one gene of PAX7, OLIG3, ATOH1, BARHL1, PTF1A and SKOR2. In addition, the dorsal metencephalic tissue induction medium may include cyclopamine in a concentration range of the SHh signaling pathway inhibitor. When the concentration of the SHh signaling pathway inhibitor is within the above range, the expression of dorsal-metencephalic region-specific genes (e.g., PAX7, OLIG3, ATOH1, BARHL1, PTF1A and/or SKOR2) is effectively greatly increased, so that the embryoid bodies may be uniformly differentiated into a cell population having dorsal-metencephalic specificity. When the concentration of the SHh signaling inhibitor is less than the above range, the expression of the dorsal-metencephalic-specific gene as described above may be minimal. In addition, when the concentration of the SHh signaling pathway inhibitor is more than the above range, the excessive concentration of the SHh signaling pathway inhibitor may inhibit cell division within the embryoid bodies, causing a problem in which the embryoid bodies do not develop as much as necessary and die.

According to one embodiment of the present disclosure, in the step of selective differentiation into the dorsal metencephalic tissue, at least one gene of PAX7, OLIG3, ATOH1, BARHL1, PTF1A, and SKOR2 may be expressed.

According to one embodiment of the present disclosure, the pluripotent stem cells may be human-derived embryonic stem cells or human-derived induced pluripotent stem (iPS) cells. The pluripotent stem cells refer to stem cells that may differentiate into three germ layers of endoderm, mesoderm, and ectoderm (pluripotency), and may include induced pluripotent stem cells (iPS) and adult stem cells, which have the capabilities, as well as the embryonic stem cells.

According to one embodiment of the present disclosure, before the step of selective differentiation into the dorsal metencephalic tissue, the method may further include a step of selective differentiation into metencephalic tissue, in which embryoid bodies formed from pluripotent stem cells are cultured in the presence of a metencephalic tissue-inducing medium containing an endogenous WNT secretion inhibitor and a WNT signaling agonist.

According to one embodiment of the present disclosure, through the step of selective differentiation into metencephalic tissue, the embryoid bodies formed from the pluripotent stem cells may differentiate into a cell aggregate that mimics metencephalic characteristics with high purity. Specifically, the cell aggregate mimicking the metencephalic characteristics may be an organoid mimicking the metencephalic characteristics with high purity. The cell aggregate or organoid mimicking the metencephalic characteristics with high purity may mean a state in which the mixing of tissues exhibiting characteristics of other brain regions such as the cerebrum, striatum, and midbrain is minimized.

According to one embodiment of the present disclosure, the step of selective differentiation into metencephalic tissue may uniformly induce patterning into metencephalic tissue throughout the embryoid bodies, while inducing differentiation of the embryoid bodies into the neuroectoderm.

According to one embodiment of the present disclosure, the endogenous WNT secretion inhibitor may be a material that inhibits the secretion of a WNT signaling material naturally expressed in the embryoid bodies. The endogenous WNT secretion inhibitor may control the heterogeneous expression of an uncontrollable WNT signaling pathway by inhibiting the activation of the WNT signaling pathway caused by a WNT protein spontaneously expressed within the embryoid bodies. Therefore, the embryoid bodies may be selectively and homogeneously differentiated into the metencephalic tissue by combining and applying the endogenous WNT secretion inhibitor and a WNT signaling agonist to be described below. The endogenous WNT secretion inhibitor may include a material capable of performing a mechanism as a Porcupine O-Acyltransferase (PORCN) inhibitor. Specifically, the endogenous WNT secretion inhibitor may include at least one selected from the group consisting of IWP-2, LGK-974, ETC-159, GNF6231, WNT-C59, and WNT974. More specifically, the endogenous WNT secretion inhibitor may include IWP-2. More specifically, the endogenous WNT secretion inhibitor may effectively neutralize the activity of the WNT signaling pathway by inhibiting the secretion of naturally expressed WNT signaling materials, and simultaneously may not affect the activity thereof even if the WNT signaling agonist is treated. That is, the endogenous WNT secretion inhibitor may effectively control the heterogeneous formation of the WNT signaling pathway by inhibiting a process of transforming the endogenous WNT protein spontaneously expressed in the embryoid bodies into a form secreted out of cells from the endoplasmic reticulum. In contrast, materials such as IWR-1-endo may have a problem of reducing an effect of the WNT signaling agonist to be described below, by inhibiting signaling in a lower cascade of the WNT signaling pathway.

According to one embodiment of the present disclosure, the metencephalic tissue-inducing medium may include an endogenous WNT secretion inhibitor in a concentration range that exhibits an effect equivalent to the effect exhibited by IWP-2 in a concentration range of 0.1 μM or more and 2 μM or less. Specifically, the metencephalic tissue-inducing medium may include an endogenous WNT secretion inhibitor in a concentration range that exhibits an effect equivalent to the effect exhibited by IWP-2 in a concentration range of 0.5 μM or more and 2 μM or less, 0.5 μM or more and 1.5 μM or less, or 0.8 μM or more and 1.2 μM or less. In addition, according to one embodiment of the present disclosure, the metencephalic tissue-inducing medium may contain IWP-2 in a concentration range of the endogenous WNT secretion inhibitor. When the concentration of the endogenous WNT secretion inhibitor is within the above range, the formation of the WNT signaling pathway spontaneously expressed in the embryoid bodies may be effectively inhibited. Specifically, when the concentration of the endogenous WNT secretion inhibitor is less than the above range, an effect of controlling the heterogeneous expression of the uncontrollable WNT signaling pathway may be minimal, resulting in the formation of a heterogeneous WNT signaling pathway. In addition, when the concentration of the endogenous WNT secretion inhibitor is more than the above range, excessive inhibition of the WNT signaling pathway may result in decreased cell division and/or decreased cell viability.

According to one embodiment of the present disclosure, the WNT signaling agonist may uniformly impart metencephalic region specificity to the entire embryoid bodies. That is, as described above, while the endogenous WNT secretion inhibitor inhibits the generation of the uncontrollable WNT signaling material in the embryoid bodies, the WNT signaling agonist may uniformly control WNT signal intensities throughout the embryoid bodies to have uniform metencephalic specificity throughout the embryoid bodies.

According to one embodiment of the present disclosure, the WNT signaling agonist may include at least one of a protein-based WNT signaling agonist and a compound-based WNT signaling agonist. Specifically, the protein-based WNT signaling agonist may include at least one selected from the group consisting of Wnt Family Member 1 (WNT-1), Wnt Family Member 3A (WNT-3a), and R-spondin-1 (RSPO1). In addition, the compound-based WNT signaling agonist may include a material that performs the mechanism of a GSK-3 beta inhibitor, and specifically, may include at least one selected from the group consisting of CHIR99021, CP21R7, CHIR98014, LY2090314, kenpaullone, AR-AO144-18, TDZD-8, SB216763, BIO, TWS-119, and SB415286. Specifically, the WNT signaling agonist may be CHIR99021.

According to one embodiment of the present disclosure, the selective differentiation into the metencephalic region may be induced by optimizing the concentration of the WNT signaling agonist in the metencephalic tissue-inducing medium. Specifically, the concentration of the WNT signaling agonist for inducing selective differentiation of the embryoid bodies into the metencephalic region may be as follows.

According to one embodiment of the present disclosure, the metencephalic tissue-inducing medium may include a WNT signaling agonist in a concentration range that exhibits an effect equivalent to the effect exhibited by CHIR99021 in a concentration range of more than 1 μM and less than 5 μM. Specifically, the metencephalic tissue-inducing medium may include a WNT signaling agonist in a concentration range that exhibits an effect equivalent to the effect exhibited by CHIR99021 in a concentration range of 1.5 μM or more and 4.5 μM or less, 1.5 μM or more and 4 μM or less, 1.5 μM or more and 3 μM or less, or 1.5 M or more and 2.5 μM or less. More specifically, the metencephalic tissue-inducing medium may contain the WNT signaling agonist at a concentration that specifically expresses at least one gene of LMX1A and LMX1B. In addition, according to one embodiment of the present disclosure, the metencephalic tissue-inducing medium may contain CHIR99021 in a concentration range of the WNT signaling agonist. When the concentration of the WNT signaling agonist is within the above range, the expression of metencephalic region-specific genes (e.g., LMX1A and/or LMX1B genes) is effectively greatly increased, so that the embryoid bodies may be uniformly differentiated into a cell population having metencephalic specificity. When the concentration of the WNT signaling agonist is less than the above range, the expression of forebrain region-specific genes (e.g., FOXG1, LHX2, and/or DLX2 genes) rather than the metencephalic region may be increased. In addition, when the concentration of the WNT signaling agonist is more than the above range, the expression of spinal cord region-specific genes (e.g., HOXB4, HOXC9 and/or HOXA1 genes) rather than the metencephalic region may be increased.

According to one embodiment of the present disclosure, in the step of selective differentiation into metencephalic tissue, at least one gene of LMX1A and LMX1B may be expressed.

According to one embodiment of the present disclosure, the step of selective differentiation into metencephalic tissue may include: a1) a step of forming embryoid bodies from pluripotent stem cells in an embryoid formation medium; and a2) a step of adding a metencephalic tissue-inducing medium containing an endogenous WNT secretion inhibitor and a WNT signaling agonist to the embryoid formation medium.

According to one embodiment of the present disclosure, the embryoid formation medium may be a liquid medium, and may be applied as long as 3D culture of the pluripotent stem cells is enabled. Furthermore, the embryoid formation medium may contain various known additives for culturing the pluripotent stem cells, depending on the purpose.

According to one embodiment of the present disclosure, the metencephalic tissue-inducing medium may include a known culture solution and additives capable of culturing embryoid bodies, and may further include the endogenous WNT secretion inhibitor and the WNT signaling agonist.

According to one embodiment of the present disclosure, the added amount of the metencephalic tissue-inducing medium may be 0.1 to 2 times greater than a volume of the embryoid formation medium. More specifically, the added amount of the metencephalic tissue-inducing medium may be equivalent to the volume of the embryoid formation medium.

According to one embodiment of the present disclosure, the step of selective differentiation into the metencephalic tissue may further include a3) a step of removing a portion of the entire medium and adding the metencephalic tissue-inducing medium at one time point during 1 to 3 days. Specifically, the step a3) may be removing the medium equivalent to the amount of metencephalic tissue-inducing medium added in the step a2) approximately every two days, and adding an equivalent amount of metencephalic tissue-inducing medium. Through this, nutrients may be supplied to the embryoid bodies, and the endogenous WNT secretion inhibitor and the WNT signaling agonist may effectively act.

According to one embodiment of the present disclosure, the step of selective differentiation into metencephalic tissue may be performed for a period of 4 to 10 days. Specifically, the step of selective differentiation into metencephalic tissue may be performed for a period of 5 to 8 days, a period of 5 to 7 days, or a period of about 6 days.

According to one embodiment of the present disclosure, the step of selective differentiation into dorsal metencephalic tissue may further include b1) a step of adding the dorsal metencephalic tissue induction medium to a medium in which the embryoid bodies are being cultured.

According to one embodiment of the present disclosure, the step of selective differentiation into the dorsal metencephalic tissue may be performed in an overlapping manner with the step of selective differentiation into metencephalic tissue. Specifically, during the period in which the step of selective differentiation into dorsal metencephalic tissue is performed in an overlapping manner with the step of selective differentiation into metencephalic tissue, the dorsal metencephalic tissue induction medium may further include the SHh signaling pathway inhibitor described above in the metencephalic tissue-inducing medium.

According to one embodiment of the present disclosure, the added amount of the dorsal metencephalic tissue induction medium may be 0.1 to 2 times greater than a volume of the culturing medium. More specifically, the added amount of the dorsal metencephalic tissue induction medium may be equivalent to the volume of the culturing medium.

According to one embodiment of the present disclosure, the step of selective differentiation into dorsal metencephalic tissue may further include b2) a step of removing a portion of the entire medium and adding the dorsal metencephalic tissue induction medium at one time point during 1 to 3 days. Specifically, the step b2) may be removing the medium equivalent to the amount of dorsal metencephalic tissue induction medium added in the step b1) approximately every two days, and adding an equivalent amount of dorsal metencephalic tissue induction medium. Through this, the nutrients may be supplied to the embryoid bodies, and the SHh signaling pathway inhibitor may effectively act. In addition, when overlapping with the step of selective differentiation into metencephalic tissue, the SHh signaling pathway inhibitor may effectively act together with the endogenous WNT secretion inhibitor and the WNT signaling agonist.

According to one embodiment of the present disclosure, the step of selective differentiation into dorsal metencephalic tissue may be performed for a period of 4 to 10 days from 3 to 5 days after the step of selective differentiation into metencephalic tissue is performed. Specifically, the step of selective differentiation into dorsal metencephalic tissue may be performed for a period of 5 to 8 days, a period of 5 to 7 days, or a period of about 6 days.

For example, when the time point of extracting and culturing the pluripotent stem cells is counted as day 0, the embryoid bodies (day 1) are formed by culturing the cells for about 1 day, and then the step of selective differentiation into metencephalic tissue may be performed from day 1 to about day 7 to about day 9, and the step of selective differentiation into dorsal metencephalic tissue may be performed from about day 3 to about day 12.

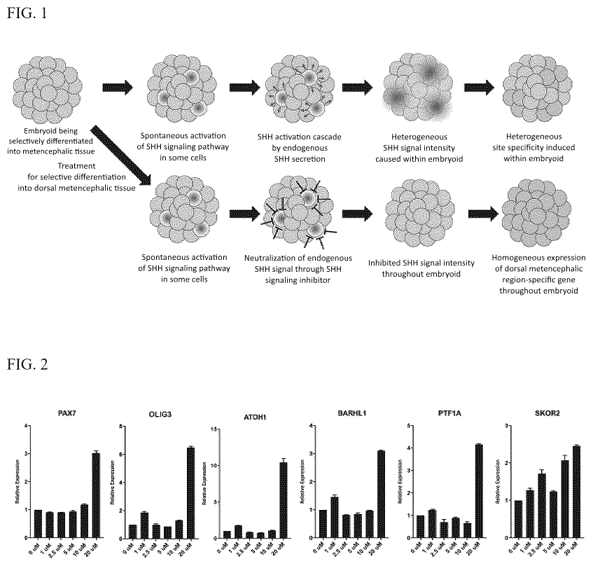

FIG. 1 is a schematic diagram showing a differentiation mechanism of a method for selective differentiation into dorsal metencephalic tissue from embryoid bodies derived from pluripotent stem cells according to one embodiment of the present disclosure. In FIG. 1, for embryoid bodies selectively differentiating into metencephalic tissue, when separate treatment is not performed, the spontaneous and heterogeneous SHh signaling pathway in some cells is activated within the embryoid bodies, thereby preventing homogeneous differentiation into dorsal metencephalic tissue (i.e., cerebellar tissue) within the embryoid bodies. In contrast, when the method for selective differentiation into dorsal metencephalic tissue according to the present disclosure is applied to embryoid bodies selectively differentiating into metencephalic tissue, the spontaneous activation of the SHh signaling pathway in the embryoid bodies may be inhibited, thereby allowing genes having dorsal-metencephalic region specificity to be uniformly expressed throughout the embryoid bodies.

The present disclosure may have various modifications and various embodiments, and specific embodiments will be hereinafter illustrated in the drawings and described in detail in the detailed description. However, the present disclosure is not limited to specific embodiments, and it should be understood that the present disclosure covers all the modifications, equivalents and replacements within the idea and technical scope of the present disclosure. In describing the present disclosure, when it is determined that a detailed description of related known arts may obscure the gist of the present disclosure, the detailed description will be omitted.

### Example 1

### [Formation of Embryoid Bodies]

Pluripotent stem cells were cultured in an embryoid formation medium as shown in Table 1 below.

TABLE 1

| Ingredient | Product name | Concentration | | Volume | |
|---|---|---|---|---|---|
| DMEM/F12 | Corning, 10-090-CV | 1X | | 37 | ml |
| KSR | ThermoFisher, 10828-028 | 1X | | 10 | ml |
| Penicillin- | ThermoFisher, 15140-122 | 100X | | 0.5 | ml |
| Streptomycin | | | | | |
| Glutamax | ThermoFisher, 35050-061 | 100X | | 0.5 | ml |
| NEAA | ThermoFisher, 11140-050 | 100X | | 0.5 | ml |
| FBS | ThermoFisher, 16000-044 | 1X | | 1.5 | ml |
| Heparin | Sigma-Aldrich, H3149 | 1 | mg/ml | 50 | ul |
| Beta- | ThermoFisher, 21985-023 | 1,000X | | 50 | ul |
| mercaptoethanol | | | | | |
| bFGF | Peprotech, 100-18B | 4 | μg/ml | 50 | ul |
| Y 27632 | Peprotech, 1293823 | 50 | mM | 50 | ul |

| | |
|---|---|
| Total | 50 ml |

When the density of pluripotent stem cells reached approximately 80%, residues on the cell surface were washed using D-PBS and separated into each cell. Furthermore, the cells were diluted to contain approximately 100,000 cells per 10 ml of an embryoid formation medium, and approximately 100 μl of the medium containing the cells was injected into each well of an Ultra-Low-attachment U bottom 96 well plate (Corning, 7007). Then, the cells were added in an incubator under a CO₂ atmosphere at 37 °C and cultured for about 24 hours to form embryoid bodies.

[Selective Differentiation into Metencephalic Tissue]

After the embryoid bodies were formed as described above, after about one day, it was confirmed whether one spherical embryoid with a clean surface was formed well in each well. A metencephalic tissue-inducing medium containing IWP-2 as an endogenous WNT secretion inhibitor at a concentration of 1 μM and CHIR99021 as a WNT signaling agonist at concentrations of 2 μM to 3 μM was prepared, and then about 100 μl of the metencephalic tissue-inducing medium was added to each well containing the embryoid bodies.

About 100 μl of the existing medium was removed once every two days, about 100 μl of the metencephalic tissue-inducing medium was added, and the embryoid bodies were cultured for 7 days to confirm whether selective differentiation of the embryoid bodies into metencephalic tissue was performed well.

As a result, it was confirmed that the specific expression of LMX1A and LMX1B genes was uniformly and well performed.

[Selective Differentiation into Dorsal Metencephalic Tissue]

A dorsal metencephalic tissue induction medium was prepared by adding cyclopamine as an SHh signaling pathway inhibitor to the metencephalic tissue-inducing medium in the concentration range of 0 μM, 15 μM, 20 μM, 25 μM, 30 μM, 35 M, 40 μM, 45 μM, and 50 μM. Then, for about 3 days from day 4 after performing the selective differentiation into the metencephalic tissue, about 100 μl of the existing medium was removed once every 2 days, and about 100 μl of the dorsal metencephalic tissue induction medium prepared as above was added, thereby simultaneously inducing selective differentiation of the embryoid bodies into metencephalic tissue and selective differentiation into dorsal metencephalic tissue.

Then, a medium was prepared excluding IWP-2 and CHIR99021 from the aforementioned dorsal metencephalic tissue induction medium. Thereafter, for about 3 days, about 100 μl of the existing medium was removed once every 2 days, and about 100 μl of the dorsal metencephalic tissue induction medium excluding IWP-2 and CHIR99021 was added, thereby inducing selective differentiation into dorsal metencephalic tissue.

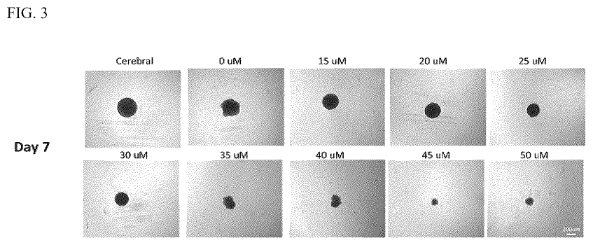

FIG. 2 shows results of expression of dorsal-metencephalic region-specific genes according to a concentration of an SHh signaling pathway inhibitor in a dorsal metencephalic tissue induction medium of Example. In addition, FIG. 3 shows a developmental state of embryoid bodies on day 7 after selective differentiation into dorsal metencephalic tissue according to a concentration of the SHh signaling pathway inhibitor in the dorsal metencephalic tissue induction medium of Example.

According to the results in FIG. 2, when the concentration of cyclopamine as the SHh signaling pathway inhibitor was 20 μM or higher, it was confirmed that the expression levels of genes PAX7, OLIG3, ATOH1, BARHL1, PTF1A, and SKOR2, capable of confirming the differentiation into dorsal metencephalic tissue, were increased rapidly. In addition, according to the results of FIG. 3, it was confirmed that when the concentration of cyclopamine as the SHh signaling pathway inhibitor was more than 30 μM, the embryoid bodies no longer grew and died. Accordingly, it was found that when the concentration of the SHh signaling pathway inhibitor in the dorsal metencephalic tissue induction medium was more than 10 μM and 30 μM or less, the embryoid bodies could be effectively cultured into dorsal metencephalic tissue.

1. A method for selective differentiation into dorsal metencephalic tissue from embryoid bodies derived from pluripotent stem cells, the method comprising:
a step of selective differentiation into dorsal metencephalic tissue, in which embryoid bodies derived from pluripotent stem cells are cultured in the presence of a dorsal metencephalic tissue induction medium containing a sonic hedgehog (SHh) signaling pathway inhibitor.

2. The method of claim 1, wherein the embryoid bodies derived from the pluripotent stem cells are embryoid bodies differentiating into metencephalic tissue.

3. The method of claim 1, wherein the step of selective differentiation into dorsal metencephalic tissue comprises a step of adding the dorsal metencephalic tissue induction medium to a medium in which the embryoid bodies derived from the pluripotent stem cells are being cultured.

4. The method of claim 3, wherein the medium in which the embryoid bodies derived from the pluripotent stem cells are being cultured includes a metencephalic tissue-inducing medium.

5. The method of claim 4, wherein the metencephalic tissue-inducing medium contains an endogenous WNT secretion inhibitor and a WNT signaling agonist.

6. The method of claim 1, wherein the SHh signaling pathway inhibitor includes at least one selected from the group consisting of cyclopamine, itraconazole, robotnikinin, cerulenin, and GANT61.

7. The method of claim 1, wherein the dorsal metencephalic tissue induction medium contains an SHh signaling pathway inhibitor in a concentration range that exhibits an effect equivalent to the effect exhibited by cyclopamine in a concentration range of more than 10 μM and 30 μM or less.

8. The method of claim 1, wherein in the step of selective differentiation into dorsal metencephalic tissue, at least one gene of PAX7, OLIG3, ATOH1, BARHL1, PTF1A, and SKOR2 is expressed.
